(19) United States
(12) Patent Application Publication
(10) Pub. No.: US 2012/0107799 A1
(43) Pub. Date: May 3, 2012

(54) DISPOSABLE, RAPID EXTRACTION APPARATUS AND METHODS

(75) Inventor: Luke T. Daum, San Antonio, TX (US)

(73) Assignee: Longhorn Vaccines & Diagnostics LLC., San Antonio, TX (US)

(21) Appl. No.: 12/916,263

(22) Filed: Oct. 29, 2010

(51) Int. Cl. C12Q 1/68; C07H 21/00; B01D 35/00

(52) U.S. Cl. 435/6.1; 422/534; 536/25.41; 422/430

(57) ABSTRACT

An extraction apparatus and methods adapted to extract and preferably also isolate structures of interest from a test sample. The extraction apparatus contains a volume-dispensing mechanism that facilitates control over the injection of a sample suspected of containing a structure of interest into a filtration vessel that contains a membrane filter that associates with the structure of interest, preferably by adsorbing or binding thereto, and a collection container associated therewith. Methods of extracting, isolating, testing, and instructions regarding such methods are also included.

### TECHNICAL FIELD

Apparatuses and methods adapted to extract or isolate a structure of interest from a sample suspected of containing the same. More particularly, the apparatus and methods facilitate rapid extraction of a structure by the contacting of a sample suspected of containing the structure to disposable filtration vessels according to the invention.

### BACKGROUND OF THE INVENTION

Separation or extraction of a component of interest from a liquid solution is important in a variety of diverse contexts, such as biological and/or chemical detection, reaction, testing, purification, concentration, and sample preparation, to name a few. This is traditionally accomplished by solid phase extraction (“SPE”) techniques in which a structure of interest is separated out of a liquid solution or mixture based on a chemical and/or physical property of a structure of interest, typically being distinct from the solvent or other material(s) present in a sample. This is often achieved by passing the liquid solution or mixture, i.e. mobile phase, over a stationary, i.e. solid, phase, resulting in the retention of the structure of interest by the stationary phase. Depending on what is being removed from the liquid solution or mixture, for example, an impurity or a purified substance, the retention by the stationary phase can be reversible.

SPE has been used in a variety of contexts for extraction or isolation of a variety of compounds in the chemical and biologic arts, such as for antibiotics, vitamins, herbicides, pesticides, fungicides, PCBs, ketones, phenols, esters, dyes, hydrocarbons, surfactants, parabens, surfactants, theophylline, oils, nucleic acids and their component bases and nucleosides, peptides, proteins, aromatic compounds, carbohydrates, aflatoxins, organic acids, nitro compounds, and drugs such as barbiturates, benzodiazepines, steroids, and caffeine, to name a few. The composition of the stationary phase depends on the ability to retain the structure of interest or the other material(s) present in the mobile (e.g., liquid phase), for example, using differences in the polarity, electrostatic interactivity or hydrophobicity/hydrophilicity of the structure of interest compared to the other material(s), the liquid phase solvent, or both.

SPE typically, although not necessarily, involves initial equilibration of the stationary phase, passage of the sample over the stationary phase, washing the stationary phase to remove one or more elements not of interest, and, if the structure of interest is not an impurity to be removed from the sample, elution of the structure of interest.

One example of the application of SPE is obtaining isolated and purified nucleic acids for a variety of diagnostic, research, preventative, and therapeutic uses. Uses of such isolated and purified nucleic acids include genetic engineering, manufacture of pharmaceuticals, medical diagnosis, identity verification, etc.

Techniques for isolating nucleic acids from cells have been used for many years. There are numerous specific methods for isolating and for purifying nucleic acids. Isolation of nucleic acids first typically involves lysing any cells or particles that are present in a sample by exposure to adverse cellular conditions. Common means include mechanical disruption, homogenization, sonication, freezing/thawing cells or exposure to hypotonic buffers, viruses, enzymes such as lysozyme, cell lysis reagents or detergents.

A second and sometimes contemporaneous step in isolating nucleic acids usually involves separating some nucleic acids from the rest of the sample. Typically, nucleic acid separation can be performed by a liquid phase extraction method, such as by the use of phenol/chloroform, salts such as chaotropic salts, or through a SPE method by adsorption of the lysed sample suspected of containing nucleic acid to a solid material, such as magnets, silica, glass fibers, Celite™, affinity resins or ion exchange chromatographs. Silica-based separation materials have been increasingly used in the laboratory and a number of commercial companies (e.g., POROS, Polymer Labs, Toso Haas, Pharmacia, PQ Corp., Zorbax, BioSepra, Amicon, Bio-Rad) sell different types, i.e. silica dioxide, borosilicate, etc., as well as different matrices, i.e. gels, resins, beads, disks, columns, wafers, etc. of this material and different varieties, i.e. amino silica, magnetized silica, chitosan-coated silica, etc.

The separated nucleic acid is then usually purified so as to be placed in the form or solution desired. Usually, in vitro manipulation and analysis of nucleic acids follows the removal from the sample of cellular and proteinaceous debris and residual compounds or contaminants present from the lysis procedure, so as not to interfere with further nucleic acid processing. A further processing step can include any type of polymerase chain reaction (“PCR”), such as real time PCR (“rPCR”), reverse transcriptase PCR (“RT-PCR”), rRT-PCR, etc., or other processing techniques.

Significant concerns with the separation and/or isolation include stability of the nucleic acids both within the cells initially and throughout the extraction process. Degradation and denaturation is a common concern, such that biological sample collection and extraction procedures are typically conducted under stringent laboratory conditions.

A variety of companies sell fairly complicated and often expensive nucleic acid extraction kits for use in the laboratory. Many commercial companies (e.g., Qiagen [Valencia, CA, ISA], OriGene [Rockville, Md., USA], and Trevigen [Gaithersburg, Md., USA]) sell nucleic acid extraction kits that exploit the use of lysis buffers and separation media in combination with the use of a microcentrifuge to separate and sometimes precipitate the nucleic acid. Some companies (e.g., Qiagen) have provided for fixed large scale purification equipment that exploits gravity flow of the nucleic acid in a lysate solution over an extraction or separation medium such as a resin or another fixed medium including those previously noted for SPE techniques. Other companies provide kits (ex., Promega [Madison, Wis., USA]) that use vacuum filtration, usually in combination with centrifugation, Several commercial companies (e.g., Qiagen, Roche [Indianapolis, Ind., USA], and bioMérieux [Durham, N.C., USA]) have developed automated or semi-automated nucleic acid extraction instruments. Others (e.g., Ambion, Invitrogen) have developed magnetic-bead based extraction kits in which the nucleic acid is separated from the rest of the solution by binding to a magnetic bead. Alternatively, some companies sell the individual components for nucleic acid extraction and rely on users to properly assemble, operate, or otherwise use such extraction equipment.

Discovery of stable, economical and rapid extraction apparatus and methods, particularly for field use or otherwise outside of a dedicated laboratory, are thus desired. Thus, the need for adaptable and quick extraction techniques in the field, such as nucleic acid isolation and purification, has been found to have increased.

### SUMMARY OF THE INVENTION

The invention encompasses a nucleic acid extraction apparatus including a filtration vessel that has at least one receiving end and that includes a membrane filter adapted to bind nucleic acids thereto, wherein the membrane filter is disposed at least substantially across a width of the filtration vessel and at least partially therein, a volume-dispensing mechanism adapted to contain and controllably dispense and forcibly inject an amount of liquid operably associated with the filtration vessel to filter the liquid therethrough, and a collection container adapted to receive the filtered liquid.

In some embodiments, the structure is a nucleic acid, including, but not limited to, deoxyribonucleic acid (DNA), ribonucleic acid (RNA), peptide nucleic acid (PNA), Morpholino and locked nucleic acid (LNA), glycol nucleic acid (GNA), or threose nucleic acid (TNA). In particular embodiments, the extraction apparatus further includes an aerosol-resistant barrier disposed on at least one side of the membrane filter.

In other embodiments, the membrane filter includes a polymer-based or silica-based matrix. In particular embodiments, the matrix includes silica fiber, silica gel, silica resin, silica-cellulose, silica dioxide, an ion-exchange resin, borosilicate, glass or diatomaceous earth, or a combination or blend thereof. In some embodiments, the ion-exchange resin includes a polymer including diethylaminoethyl-, quaternary aminoethyl- or quaternary ammonium-groups.

In some embodiments, the filtration vessel includes polyethylene, linear polyethylene, low density polyethylene, polypropylene, polystyrene, polycarbonate, endotoxin-free or pyrogen-free plastic, or a combination or a blend thereof.

In other embodiments, the extraction apparatus further includes a flow valve positioned between the liquid dispensing mechanism and the filtration vessel to limit fluid flow to one direction.

In some embodiments, the extraction apparatus further includes a substantially leak-free connector positioned between the liquid dispensing mechanism and the membrane filter.

In particular embodiments, the liquid dispensing mechanism includes a piston pump, syringe, pipette, micropipette, bulb, or dropper.

In yet other embodiments, the membrane filter of the extraction apparatus is restricted from movement by a tether including at least one gasket, O-ring, indentation of an opposing inner surface of the filtration vessel, adhesive, or a frit, or a combination thereof.

In some embodiments, the collection container of the extraction apparatus releasably attached at an end of the filtration vessel.

In further embodiments, the extraction apparatus further includes a replaceable cartridge operably associated with the volume dispensing mechanism and the filtration vessel. In some embodiments the replaceable cartridge contains a prepared sample suspected of containing a structure of interest, a wash solution, or an elution buffer. In yet other embodiments, the apparatus further includes one or more fittings, adapters, connectors, valves, or a combination thereof.

In still further embodiments, the extraction apparatus according to the invention is adapted to operate in the absence of electricity, microcentrifuge, or a vacuum manifold device, or even all of these combined.

The invention also encompasses a method for extracting a structure of interest from a liquid, which includes: preparing a sample suspected of containing a structure of interest for passage through a filtration zone, and dispensing a portion of a prepared sample suspected of containing a structure of interest through a filtration zone by applying and controlling injection pressure applied to the sample.

In some embodiments, the method further includes washing the filtration zone to remove one or more impurities therefrom. In other embodiments, the method further includes eluting at least a portion of the structure of interest from the filtration zone. In some embodiments the sample is a biological sample including blood, plasma, cell, tissue, or serum, or any combination thereof In yet further embodiments, the structure of interest is a nucleic acid including, but not limited to, deoxyribonucleic acid (DNA), ribonucleic acid (RNA), peptide nucleic acid (PNA), Morpholino and locked nucleic acid (LNA), glycol nucleic acid (GNA), or threose nucleic acid (TNA).

In particular embodiments, at least a portion of the sample is dispensed through the filtration zone at least twice prior to the washing and eluting.

In some aspects of the invention, the eluted second portion of the nucleic acid is sterilized.

In other embodiments, the method further includes preparing the sample for dispensing by first associating the sample with a composition including one or more: chaotropes, detergents, reducing agents, chelators, buffers, or a combination thereof. In related embodiments, preparing the sample includes lysing the sample suspected of containing nucleic acids.

In particular aspects of the invention, the method further includes washing the filtration zone at least twice before eluting the second portion of the nucleic acid therefrom.

In some embodiments, the eluting includes applying TE buffer, water, tricine, bicine, or Tris-HCl, or a combination thereof.

In other embodiments, the extracting is performed a temperatures from about 10° C. to about 40° C. In particular embodiments, the extracting is performed within up to about 60 minutes, preferably 30 minutes, more preferably, 10 minutes or 5 minutes, even yet more preferably within about 2 to 3 minutes, or within about 1 minute of placing the sample in the extraction vessel. In some instances, the method further includes detecting or characterizing the nucleic acid.

In some embodiments, the portion of the prepared sample is a pre-calibrated unit amount thereof. In yet other embodiments, the dispensing includes operating the plunger to pressurize the sample.

The invention further includes a field extraction kit, in suitable container means, including: the above-mentioned extraction apparatus and self-contained reagents including one or more choatropes, detergents, reducing agents, chelators, buffers, or a combination thereof.

The invention further encompasses a set of instructions to carry out any of the methods discussed herein, along with the extraction apparatus, filtration vessel, volume-dispensing mechanism, or the collection container, as applicable, operably associated with the instructions.

### BRIEF DESCRIPTION OF THE DRAWINGS

The present disclosure can be better understood from the following detailed description when read with the accompanying figures. It is emphasized that various features are not drawn to scale and are used only for illustrative purposes. In fact, the dimensions of the various features may be independently increased or reduced.

FIG. 1 illustrates an exploded view of an embodiment of the invention;

FIG. 2 illustrates an exploded view of another embodiment of the invention;

FIG. 3 illustrates a plan view of an embodiment of the invention;

FIG. 4 illustrates an exploded view of an embodiment of the invention;

FIG. 5 depicts the resultant amplicon from RT-PCR analysis and gel electrophoresis of H3N2 influenza RNA; the RNA being extracted from a previously tested Influenza Virus subtype H3N2 positive human clinical sample using the extraction tip embodiment of the invention (as depicted in FIG. 3), compared to a ladder and no template control included on the gel;

FIG. 6 shows the extraction efficiency of an embodiment of the invention compared to a commercial extraction column kit (Ambion, Austin, Tex.) with extraction efficiency evaluated using the ABI 7500 (Applied Biosystems) with the comparative Cycle Threshold (C_{T}) method;

FIG. 7 shows that extraction efficiency increases when a sample containing nucleic acid is passed through an apparatus of the invention (as depicted in FIG. 2) multiple times;

FIG. 8 shows the extraction efficiency of an apparatus of the invention with Influenza Virus subtype H3N2 RNA obtained from a cultured stock, as well as an Internal Positive Control (IPC) to distinguish false positives and negatives from true ones; and

FIG. 9 shows the extraction efficiency of Influenza Virussubtype H1N1 RNA using an embodiment of the invention as depicted in FIG. 1.

### DETAILED DESCRIPTION OF PREFERRED EMBODIMENTS

The apparatus and methods described herein facilitate accurate and rapid extraction of a structure of interest via a stable setup that contains a filtration vessel that includes a filter (preferably a membrane-type filter), a volume-dispensing mechanism, and a collection container. Exemplary embodiments of the invention are discussed herein, and it should be understood that various embodiments may be substituted for each other, or used in addition to each other, in appropriate circumstances. In this specification, the extraction apparatus is sometimes referred to as an “extraction vessel” or “extraction zone.” The invention can advantageously permit extraction in a matter of minutes, rather than hours as conventionally required. In one preferred embodiment, the extraction apparatus is handheld, i.e., portable, although the collection vessel or zone may be coupled thereto, or may rest on a surface and be operatively associated with the remainder of the extraction apparatus. For example, the present methods may be carried out to extract nucleic acids from a sample less than about 60 minutes, preferably less than about 30 minutes, more preferably less than about 10 minutes or about 1 to about 10 minutes, and even more preferably in less than 1 minute after placing the sample in the extraction vessel. In preferred embodiments, the extraction (including repeated passes) can be achieved in less than about 5 minutes, preferably less than about 3 minutes (e.g., 2-3 minutes), and in certain embodiments less than about 2 minutes or about 1 minute after placing the sample in the extraction vessel.

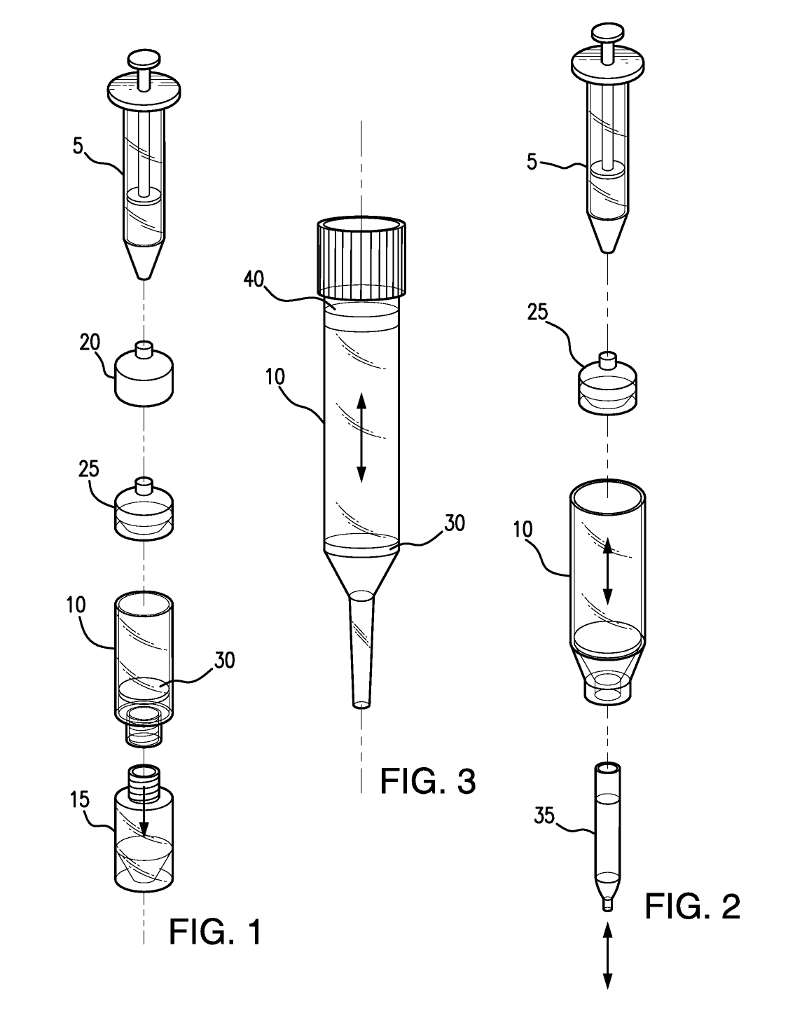

FIG. 1 illustrates an exploded view of a preferred embodiment of an extraction vessel according to the invention. The extraction vessel in this embodiment includes a volume-dispensing mechanism 5, in which a sample suspected of containing a structure of interest is initially placed, a filtration vessel 10, in which a membrane filter 30 adapted to retain a portion of the structure of interest is placed, and a collection container 15, adapted to receive any filtrate after the sample, wash or buffer, such as an elution buffer, is passed through the filtration vessel. Preferably the entire extraction vessel is disposable once the analyte extraction procedure is completed. Optionally, the extraction vessel can be reversibly mounted onto a supporting stand so as to enable ease of use. The portion retained on the filter 30 may be a majority, preferably at least substantially all, more preferably all but a trace amount, of the structure of interest (or the materials being separated from such structure of interest). The filter 30 may operate through any technique available to those of ordinary skill in the art, including a physical filter, adsorption, binding, or other technique. The depicted embodiment shows a flow path directly from the volume dispensing device through the filtration device and to the extraction vessel. The flow path can be at least substantially linear in a preferred embodiment, although it can instead be curved or otherwise be bent or angled, or any combination of the above, if needed to address overall size or other issues.

The volume-dispensing mechanism 5, the filtration vessel 10, and the collection container 15, may be integrally molded to one another or placed in such a way that at least one of the components may be disconnected from the other parts of the extraction vessel. It is preferable that the collection container 15, be detached or detachable from the other components of the extraction vessel and able to be readily replaced, so that waste solutions and solutions containing the structure of interest can be collected separately. This can also facilitate reuse of the filtration vessel, such as after washing and replacement with a clean filter 30, a cleaned or different collection container 15, and a volume-dispensing mechanism 5. Although different sized components can be used, each of which can be independently selected having a different or the same diameter as other components, the components will typically have a diameter ranging from about 0.25 cm to 2 cm, preferably about 0.5 cm to 1.25 cm.

The volume-dispensing mechanism 5, contains at least one opening through which the liquid sample and air can flow. Any liquid or gas, i.e. sample, wash buffer, elution buffer, can enter or charge the volume-dispensing mechanism 5 by the displacement of air within the volume-dispensing mechanism 5. A sample of interest can be poured or otherwise disposed into the volume-dispensing mechanism 5. Then, an amount of specimen can be transferred to the filtration vessel 10 for extraction and preferably also isolation of the structure of interest from other components within the specimen. In either case, the transfer of specimen is typically accomplished by the application of force or pressure to forcibly inject the gas or liquid into the filtration vessel 10. Typical, volume-dispensing mechanisms 5 can include, but are not limited to, one or more of a piston pump, a syringe, pipette, micropipette, or dropper. Each can be disposable after single-use, or be adapted for repeated use or repeated use after proper cleaning, sterilization, or both.

Preferably, the volume-dispensing mechanism 5 allows manual control of the flow rate, quantity, or both, of the liquid being dispensed into the filtration vessel 10. The flow rate should preferably be sufficient to force the sample or specimen to and through the membrane filter 30. The volume-dispensing mechanism 5 can dispense various volumes of liquids and can be labeled with graduated marks to indicate volume amounts. Alternatively, the volume-dispensing mechanism can be pre-set so as to draw up a predetermined amount of fluid. Preferably, the volume-dispensing mechanism 5 can repeatedly dispense a volume amount anywhere from about 1 μl to about 100 mL, more preferably 50 μl to about 50 mL and most preferably 100 μl to about 20 mL. The volume-dispensing mechanism 5 can be used to repeatedly dispense the same or different samples, or quantities of the same sample, or the same or different samples at different flow rates. The portion of the volume-dispensing mechanism 5 that is in contact with the sample is preferably made from material(s) that do not substantially bind the structure of interest. For example, when extracting nucleic acids, the portion of the volume-dispensing mechanism 5 that is in contact with the sample suspected of containing nucleic acids can be made of a polymer component, such as polypropylene and polyethylene, or a blend thereof, or latex, rubber, bis(2-ethylhexyl)phthalate (DHEP), and oil-free silicone, or a combination thereof. The volume-dispensing mechanism 5 may take any shape to allow for dispensing of a sample. Preferably, the volume-dispensing mechanism 5 is cylindrical or otherwise rounded (e.g., oval, elongated), and the portion of the volume-dispensing mechanism 5 nearest the receiving end of the filtration vessel 5 preferably has a conical or otherwise tapered shape, i.e. progressively reduced in the inner diameter, so that pressure increases as the sample exits the volume-dispensing mechanism 5. This can advantageously provide increased granular control over the quantity dispensed, and can help ensure the sample contacts the filter 30 first. It can also permit structural components to be disposed to facilitate connection of the volume-dispensing mechanism 5 and the filtration vessel 10, for example, using a rubber stopper with a hollow inner diameter that is sized to admit a portion of the tapered end of the volume-dispensing mechanism 5 and an outer diameter that is sized and shaped to securely fit around or into an intake end of the filtration vessel 10.

The volume-dispensing mechanism 5 operably associates with the filtration vessel 10. Any sample that is contained in, passes through, charges or is otherwise placed into the volume-dispensing mechanism 5 can be transferred into the filtration vessel, e.g., via the application of pressure to the volume-dispensing mechanism 5. The volume-dispensing mechanism 5 can either connect to the filtration vessel 10 or connect to valves, which then connect or attach to the filtration vessel 10. The opening that allows fluid to pass out of the volume-dispensing mechanism preferably has an inner diameter such that the volume-dispensing mechanism 5 can fit within or around a receiving end of the filtration vessel 10, one or more valves in an air-tight manner, or press-fitted over the tip of the filtration vessel 10 or one or more valves. A liquid-tight connection should preferably exist between the volume-dispensing mechanism 5 and any connected portion of the extraction vessel, i.e. a receiving end of the filtration vessel 10, or the one or more valve(s). To accomplish this, any type of suitable fitting may be placed on the end of the volume-dispensing mechanism 5, the filtration vessel, or the valve(s), such as one or more threaded connections, or leak-free connectors such as Luer tapers, Luer-Loks, and Luer-Slips. This may include an operably associated filtration vessel cap, which may contain or be integrally or releasably associated with the valve(s). Alternatively, the valve(s) can be placed within the filtration vessel 10 itself near the end closest to the volume-dispensing mechanism 5. When a filtration vessel cap is used, it can be releasably or permanently joined to the filtration vessel 10 by any available device, including but not limited to a snap, switch, locking, or screw arrangement, or a combination thereof.

The inlet valve 20 near or at the receiving end can regulate the flow of the sample from the volume-dispensing mechanism 5 to the filtration vessel 10. A check or non-return valve 25 may also preferably be present to prevent the back flow of sample into the volume-dispensing mechanism 5, allowing the sample to pass in one direction only. Preferably, the one or more valves are made of a material that does not substantially bind, or preferably does not bind more than a trace amount, of the structure of interest and preferably of any other components in the sample. This material can include one or more thermoplastic polymers, for example, polymers such as a C₁-C₂₀ polyolefin, preferably a C₁-C₈ polyolefin, or a combination or blend thereof. Preferably, the polymers will be formed from ethylene or propylene, or a combination or blend thereof. In some embodiments, each polyolefin present can include more complex olefin branches, can include double bonds, can contain substitutions with functional groups, or a combination thereof.

In the above-noted embodiment, as well as certain others, the filtration vessel 10 will have a discharge opening through which liquid will flow after passing over the membrane filter 30, in addition to the receiving end. In other embodiments, however, the receiving end may be arranged to also function as a discharge opening after sufficient charging of the filtration vessel 10 and filtration of the structure of interest has been achieved. A switch or lever, for example, might be disposed so a user can move any one-way valve off-line after filtration to permit the fluid to flow back the same way it entered. In such an embodiment, the volume-dispensing mechanism will typically have been replaced with, or also adapted to act as, a collection device for the captured structure(s) of interest.

The filtration vessel 10 preferably has at least two openings, a receiving end and a discharge opening. At least one of the openings, the receiving end, receives the sample from the volume-dispensing mechanism 5, thereby at least partially charging, preferably at least substantially charging, the filtration vessel 10 with the sample and at least partially discharging, preferably at least substantially discharging, the volume-dispensing mechanism 5, preferably after the sample is passed through one or more valves 20, 25. The discharge opening functions as an outlet for the filtration vessel 10 and allows for the passage of a portion of a purified sample to be passed through to the collection container 15. The filtration vessel 10 typically contains a corresponding amount compared to the amount initially dispensed by the volume dispensing mechanism 5. Preferably, the filtration vessel 10 can contain volumes of about 0.5 mL to about 5 mL. Inside the filtration vessel is one or more membrane filters 30 to which the structure of interest in the liquid preferentially adsorbs or binds to.

The membrane filter 30 may be made of any material available to those of ordinary skill in the art to which nucleic acids preferentially adsorb or bind to, such as silica, nitrocellulose, polyamide, nylon, glass, diatomaceous earth, magnetic carriers, anion-exchange material, or any combination or blend within or between any of these categories of binding materials. Preferably, the membrane filter 30 includes a polymer-based or silica-based matrix. The matrix can be a wafer or any suitable shape of material, such as gels, resins, beads, disks, columns, microspheres and can be paper-backed with matrix material on one side or both sides and if present on the one side, could be located upstream or downstream of the backing. Preferably, the matrix includes silica fiber, silica gel, silica resin, silica-cellulose, silica dioxide, an ion-exchange resin, borosilicate, glass fibers, quartz wool, or diatomaceous earth, or a combination or a blend thereof. In a preferred embodiment, the membrane filter 30 can include borosilicate or silica dioxide, preferably in a compressed fiber arrangement. If the membrane filter 30 is made of an ion-exchange resin, it preferentially includes a polymer comprising diethylaminoethyl-, quaternary aminoethyl- or quaternary ammonium-groups. In one preferred embodiment, borosilicate fibers disposed on a porous substrate (commercially obtained from, for example, Advantec MFS, Inc., Pleasanton, Calif.) that are about 0.1 to about 0.7 mm, preferably about 0.4 mm to about 0.5 mm, thick, are cut into circles with diameters of about 0.1 cm to about 3 cm, more preferably about 0.5 cm to about 1.25 cm, and placed inside the filtration vessel 10. The substrate can include or be any porous material, such as a matrix, grid, layered arrangement, or the like. The substrate can include paper, for example. The substrate, and in preferred instances, the adsorbent or binding material, is preferably sized and dimensioned to fit in the filtration vessel so as to contact an inner edge of the filtration vessel to minimize sample from passing the membrane filter 30 without being filtered.

The membrane filter 30 may be held in place by any acceptable device or technique that allows for a portion of the structure of interest to bind to the membrane filter 30, while substantially the rest of the sample passes through to the collection container 15. Preferably, any structure or device associated with the membrane filter 30 to retain it at least substantially in place will not permit the structure of interest to substantially bind to the structure or device. This can include the use of at least one gasket, wafer, O-ring, indentation of an opposing inner surface of the filtration vessel, adhesive, or a frit, or a combination thereof The membrane filter 30 is disposed at least substantially across, preferably all the way across, a width (and preferably all widths) of the filtration vessel 10 and at least partially, preferably entirely, therein. Preferably, a seal is formed between the inner wall of the filtration vessel 10 and the holding device to retain the membrane filter 30 at least substantially in place so as to minimize or preferably avoid leaking of the sample around the membrane filter 30. Preferably, the membrane filter 30 is placed closer to, near, adjacent, or at, the end of the filtration vessel 10 closest to the collection container 15 so as to provide sufficient volume for fluid to reside in the filtration vessel 10 between it and the volume-dispending mechanism 5.

The filtration vessel 10 is operably associated with the collection container 15. Typically, the collection container 15, filtration vessel 10, and any operably associated valves will collectively measure from about 1 cm to about 15 cm, preferably about 2 cm to 10 cm, and more preferably about 3 cm to about 8 cm, in length when assembled. The collection container 15 is adapted to collect any remaining sample after the sample has contacted, and preferably passed through, the membrane filter 30. The collection container 15 can also collect any waste material that results from washing the structure-bound membrane filter 30. The wash may be any available washing material, including but not limited to one or more alcohols, acetates, or a combination thereof. Preferably, the contents of the collection container 15 including lysed sample and subsequent washes are discarded after passage through the membrane filter 30, or after a plurality of passes for a given sample. In a preferred embodiment, the collection container 15 can reversibly attach to the filtration vessel 10 by any mechanism available to those of ordinary skill in the art, including but not limited to threading, switch, snap, a locking device or a securing device, or any combination thereof. The collection container 15 typically contains a corresponding amount compared to the amount initially dispensed by the volume dispensing mechanism 5 and the filtration vessel 10. Preferably, the collection container 15 can contain volumes of about 0.5 mL to about 5 mL. The corresponding amount may be greater or lesser than the initial amount depending on any amounts retained by or released from other extraction apparatus component(s). Preferably, the collection container is made of easily disposable materials, such as one or more biodegradable plastics.

Alternatively, a separate collection container 15 can be used when the structure of interest is eluted from the membrane filter. In a preferred embodiment, the collection container 15 containing eluted nucleic acid is a sterilized microcentrifuge tube, although any suitable vessel of appropriate dimensions and materials based on the guidance herein may be used.

With reference to the various alternatives and embodiments above, FIG. 2 illustrates an exploded view of another preferred embodiment of an extraction vessel. The extraction vessel in this embodiment includes a volume-dispensing mechanism 5, in which a sample suspected of containing a structure of interest is initially placed or passed through from a source, a filtration vessel 10, in which a membrane filter adapted to bind or adsorb to a structure of interest is placed, a collection container 15 (not shown) and a tip 35 which allows for the two-way flow of liquid back through the tip 35 and into the filtration vessel 10 so that at least some, preferably most, more preferably substantially all, the liquid can pass back and forth across the membrane filter 30 repeatedly to increase the purification of the structure of interest. In this embodiment, the collection container 15 is preferably disposed so the tip 35 is adapted to extend therein for depositing and withdrawing additional sample for further passes through the membrane filter 30. Typically, the filtration vessel 10 and any operably associated valve(s) 20 measures about 1 cm to about 8 cm in length, preferably about 1.5 cm to about 6.5 cm, and more preferably about 2 cm to about 5 cm in length. The tip 35 can be, to a large extent, made of any material available to those of ordinary skill in the art that doesn't substantially bind to the structure of interest, such as, where a nucleic acid is the structure of interest, any thermoplastic polymer component independently selected from among those listed above. The tip 35 contains at least two openings through which sample liquid can flow—from the filtration vessel 10 to the collection container 15 and back. This can be achieved through engagement of the volume-dispensing mechanism 5 with the filtration vessel 10, and the volume-dispensing mechanism can be engaged and disengaged to pull fluid sample back from the collection container 15 through the tip 35 to the filtration vessel 10 when the volume-dispensing mechanism 5 is released or otherwise used to create suction. The tip 35 preferably reversibly attaches to the filtration vessel 10 by any reasonable mechanism such as threading, switch, snap, a locking device or a securing device, or any combination thereof. Preferably, the portion of the tip 35 nearest the opening that allows fluid samples to pass out of the tip 35 to the collection container 15, has a rounded shape, preferably conical or tapered, i.e. progressively reduced in the inner diameter, so that pressure increases as the sample exits the tip 35. This can advantageously facilitate control over the amount of sample discharged or suctioned up, as well as better controlling the flow rate of the sample during this procedure. Preferably, the tip 35 measures about 1 to about 15 cm in length, more preferably, about 2 cm to about 10 cm in length. Preferably, the tip 35 is disposable once the extraction procedure is complete, which can, for example, minimize contamination of different samples without need for disposing of the entire extraction vessel after each use.

FIG. 3 illustrates a plan view of a preferred embodiment of an extraction vessel. The extraction vessel in this embodiment includes a volume-dispensing mechanism (not shown), in which air is displaced, a filtration vessel 10, in which a membrane filter 30 adapted to bind or adsorb to a structure of interest is placed, and a collection container 15 (not shown) is disposed to collect the remaining sample that does not bind or adsorb, or both, to the one or more membrane filters 30. Preferably, the filtration vessel 10 reversibly attaches to the volume-dispensing mechanism by any reasonable device available to those of ordinary skill in the art, including but not limited to threading, a switch, a snap, a locking device or a securing device, or any combination thereof. Preferably, the membrane filter 30 is operatively associated with an aerosol resistant barrier 40 that inhibits or entirely blocks contamination of the volume-dispensing mechanism. In one embodiment the aerosol resistant barrier 40 is placed on at least one side of the membrane filter 30. Preferably, the aerosol resistant barrier 40 is placed within the filtration vessel 10 between the membrane filter 30 and the end of the filtration vessel 10 closest to the volume-dispensing mechanism. In another embodiment, the aerosol resistant barrier 40 is disposed adjacent to the membrane filter 30. In a preferred embodiment, the aerosol resistant barrier 40 is disposed adjacent the end of the filtration zone or vessel that connects with the volume-dispensing mechanism 5. In yet another embodiment, the aerosol resistant barrier 40 is disposed on both sides of the membrane filter 30. The aerosol resistant barrier 40 can be held in place by the nature of the material itself, i.e. is self-sealing or self-adhering, or by any other reasonable means such as at least one gasket, O-ring, indentation of an opposing inner surface of the filtration vessel, adhesive, or a frit, or a combination thereof Preferably, the aerosol resistant barrier 40 is made of a hydrophobic material. Preferably, the filtration vessel 10 with aerosol barrier 40 is autoclavable before the membrane filter 30 is placed in the filtration vessel 10, which can advantageously permit sterilization of the aerosol barrier 40 before reuse for further collection activities.

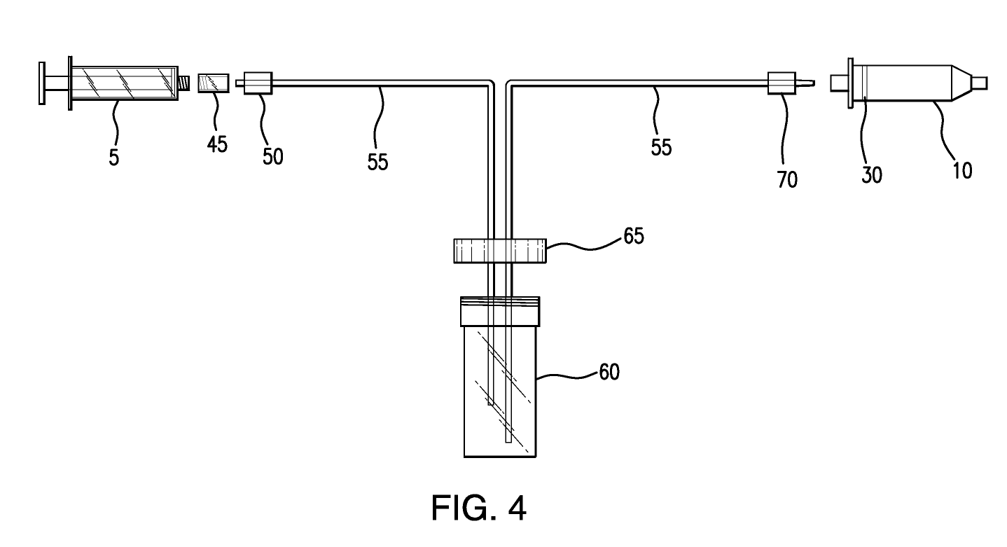

With reference to the various alternatives and embodiments herein, FIG. 4 illustrates an exploded view of another preferred embodiment of an extraction apparatus. The extraction apparatus in this embodiment includes a volume dispensing mechanism 5 in which air or another gas (or other non-reactive fluid that does not chemically or biologically affect the sample) is displaced by an operator, a replaceable cartridge 60 into which either an amount of (i) a prepared sample (typically a liquid) suspected of containing a structure of interest, (ii) a wash or (iii) an elution buffer is placed, and a filtration vessel 10 containing a membrane filter 30. A plurality of such replaceable cartridges can be prepared in advance and even operatively associated with the extraction apparatus if desired, particularly with wash or elution buffer. In a preferred embodiment, the components of the extraction apparatus are adjacent to, but not in direct alignment with one another, and are instead aligned so as to facilitate removal and replacement of the replaceable cartridge 60 from and to the rest of the extraction apparatus. In this embodiment, there is a fluid flow path from the volume dispensing mechanism 5 through the replaceable cartridge 60 through the filtration vessel (or zone) 10 to the collection vessel. In the depicted embodiment, the replaceable cartridge 60 is adjacent to, but not along, a direct path from the volume dispensing mechanism 5 and the filtration vessel 10. Preferably, tubing 55 connects the volume dispensing mechanism 5 to the replaceable caitiidge 60 and the replaceable cartridge 60 to the filtration vessel 10.

In one embodiment, the volume dispensing mechanism 5 connects directly to the replaceable cartridge 60, thereby avoiding the use of tubing 55 (not depicted in FIG. 4) connecting the volume dispensing mechanism 5 and the replaceable cartridge 60. In another embodiment, the replaceable cartridge 60 connects directly to the filtration vessel 10, thereby avoiding the need for tubing 55 (not depicted in FIG. 4) connecting the replaceable cartridge 60 and the filtration vessel 10. In yet another embodiment, tubing 55 is absent from the extraction vessel. In some embodiments, the tubing may be sufficiently rigid to maintain its shape between any of the extraction apparatus components if desired. In any case where tubing is present, it may include any material or substance that does not substantially bind, or bind at all, to the structure of interest. For example, when the extraction vessel is used to extract nucleic acids, the tubing may be microfluidic tubing composed of Tygon® tubing. Preferably, the tubing is able to be sterilized prior to use. Tubing length and diameter may be of any size, depending on the pressure needed and the size of the openings necessary to connect the replaceable cartridge 60 with one or more other components of the extraction apparatus. The tubing 55 may be connected to each of the volume dispensing mechanism 5, the replaceable cartridge 60, or the filtration vessel 10 by any type or number of suitable fittings, adapters, connectors or valves such as leak-free connectors such as Luer tapers, Luer-Loks, and Luer-Slips. Preferably, the use of these creates a liquid-tight connection, more preferably also an air-tight connection, between the components of the apparatus.

FIG. 4 illustrates some of these components, i.e., a Luer taper 45, a fitting 50, and a Leur lock connector 70. In some embodiments, a bonding material or adhesive may be included to better secure the tubing 55 to the one or more components, including the fittings, adapters, connectors or valves. Such bonding material or adhesive is preferably non-reactive with the elution buffer, wash, and sample materials that will be placed inside during use. The valves may be inlet valves, check valves, or non-return valves as described herein, and can be used to limit contamination of various portions of the apparatus through backwash from the next portion in the fluid flow path. The replaceable cartridge 60 is adapted to be rapidly removed or added from or to other components of the extraction vessel so that various compositions can be used and/or pumped within the extraction apparatus without need to wash, clean, or otherwise replace the volume dispensing device 5. The replaceable cartridge 60 may be self-sealing around other components such as the tubing 55, the filtration vessel 10 or the volume dispensing mechanism 5. The replaceable cartridge 60 may be composed of any material that does not substantially bind, or bind at all, to the structure of interest. For example, when nucleic acids are being extracted using the extraction vessel, the replaceable cartridge 60 may be formed from a centrifuge tube. Of course, the apparatus functions without need for centrifugation of the sample or extraction apparatus.

In some embodiments, a top 65 is operably associated with the replaceable cartridge 60 and can be releasably or permanently joined to the replaceable cartridge 60 by any connection device available to those of ordinary skill in the art, including but not limited to a snap, switch, lock, clamp, clamp, safety lever, bar, thread or screw arrangement, or any combination thereof, or otherwise quickly secured into place or removed when desired, through any technique available to those of ordinary skill in the art through a relatively fixed point at which the other components of the extraction apparatus can be gathered and presented for rapid connection and replacement of such a replaceable cartridge 60. Preferably, the replaceable cartridge 60 is releasably joined. The connection device can, of course, attach one or more components of the extraction apparatus to any portion of the replaceable cartridge 60, such as the side or bottom thereof and is not limited to the top 65. The top 65 may also be releasably or permanently joined to the tubing 55 to facilitate fluid flow from the volume dispensing device therethrough to the filtration vessel or zone. Preferably, the top 65 is sterilized or a new top 65 is associated with a new replaceable cartridge 60 between uses of the extraction vessel so that only the replaceable cartridge 60 needs to be changed when extracting multiple samples with the same extraction vessel. For example, the replaceable cartridge 60 may contain a prepared sample suspected of containing a structure of interest. Once that sample is dispensed into the tubing 55 (or other connection) connecting the replaceable cartridge 60 and the filtration vessel 10 by dispensing some of the fluid in the volume dispensing mechanism 5 associated with the replaceable cartridge 60, another replaceable cartridge containing a wash solution and/or an elution buffer may be swapped in place of the sample-containing replaceable cartridge, and then connected and dispensed in a similar manner. The wash solution may be used to wash the membrane filter 30 and/or to wash one or more components of the extraction vessel. The filtration vessel 10 is again operably associated with at least one collection container (not shown in FIG. 4) to capture any solution dispensed from the filtration vessel 10 after the compositions have passed over the membrane filter 30. Preferably, all components of this embodiment are interchangeable, and can be disposed of and replaced when needed or desired, although preferably the volume dispensing mechanism 5, fittings, adapters, connectors, valves, and tubing 55, can be each sterilized if needed or desired, and then reused.

A sample suspected of containing a structure of interest can be collected and pre-treated in any number of ways before the extraction apparatus of the present invention is used. For example, samples can be diluted, maintained at certain temperatures or at a certain pH or combined with certain chemicals so as to preserve the configuration of the structure of interest—or to modify it, if desired, such as to inactivate any living portion of the sample or any portion of the sample that would degrade or interfere with the structure of interest. Preferably, the sample is maintained to prevent substantial degradation or deterioration of the structure of interest. In addition, there may be additional extraction before the sample enters the extraction vessel. Likewise, the filtration zone, or area where the structure of interest is separated from the rest of the sample, may need to be pre-treated so that it responds appropriately when sample is passed through.

Once the sample suspected of containing the structure of interest is obtained and prepared, the sample is applied to the extraction apparatus. The sample is taken up and dispensed into a filtration zone, preferably by the application of pressure to the volume-dispensing mechanism. Alternatively, temperature modification or other mechanisms could be used to help urge sample into contact with the filtration zone, such as one or more membrane filters 30 in the filtration zone. After the sample passes through the filtration zone, the structure of interest preferably remains retained within the filtration zone by adsorbing or binding to a filtration material. It should be understood that various physical or chemical phenomena, or both, may cause binding, such as electrostatic charge, ionic bonding, covalent bonding, or the like, or any combination thereof. Preferably, the binding or adsorption is reversible so that the retained substance can be removed and the membrane filter 30 reused or recycled if feasible. The filtration zone may be exposed multiple times to the same or different samples. The filtration zone may also be washed with, for example, a wash solution or buffer, to ensure that substantially no other material, besides the structure of interest, remains in the filtration zone. The filtration zone may be washed multiple times. Typically most, preferably substantially all, of the structure of interest contained in the sample is adsorbed or bound in the filtration zone. If the structure of interest is an impurity, the purified sample is collected and the extraction vessel with the impurity adsorbed or bound to it is dispensed into the collection zone. If the structure of interest is needed for further analysis, the structure of interest can be eluted from the filtration zone by any available technique available to those of ordinary skill in the art, including but not limited to application of an appropriate liquid or gas solution. Typically a majority, and preferably at least substantially all, of the structure of interest retained in the filtration zone is eluted. In one embodiment, the eluted structure of interest is substantially or essentially free, more preferably entirely free, of other components that were originally contained in the sample. Once eluted and isolated, the purified sample or the structure of interest, or both, can be subjected to further conventional analytical procedures and methods as would be known by one of ordinary skill in the art.

The methods according to the invention can be used to isolate or extract a variety of structures including, but not limited to, one or more: antibiotics, vitamins, herbicides, pesticides, fungicides, PCBs, ketones, phenols, esters, dyes, hydrocarbons, surfactants, parabens, surfactants, theophylline, oils, nucleic acids and their component bases and nucleosides, peptides, proteins, aromatic compounds, carbohydrates, aflatoxins, organic acids, nitro compounds, and drugs such as barbiturates, benzodiazepines, steroids, caffeine, or any combination thereof Preferably, the method is used to isolate structures from a variety of sample sizes, more preferably this method is used for isolating analytes from smaller sample volumes and can detect small amounts of analytes in a sample. Typically, the extraction apparatus and methods of the invention will be advantageous for field use, particularly in rural or other environments remote from modern laboratory facilities, such as in the desert, in third world countries, in crisis-struck regions that have no steady electricity supply, in triage centers, at border crossings, or the like. Preferably, the apparatus is not adapted to use, and the methods exclude, the use of electricity. In various preferred embodiments, the apparatus and methods are not adapted to include a centrifuge, vacuum manifold, incubator, heat bath, physical agitation, or vortexor, or any of these devices, to achieve extraction. Any of these can be used on a sample before or after extraction, often at a different location, but the extraction itself preferably does not require use of any of these devices. In a preferred embodiment, the extraction apparatus and methods can be used at the point of care for detection during an epidemic, pandemic, or outbreak, such as of influenza or another biological agent.

The methods are particularly beneficial in isolating one or more biological molecules of interest. Samples can be prepared according to any process available to one of ordinary skill in the art. In some instances, samples can be prepared by or during the process of collecting the sample. In other embodiments, the collected sample is additionally treated in preparation for filtration. For example, once a sample suspected of containing a nucleic acid of interest is obtained, it is usually freed from the proteins, carbohydrates, lipids and/or other nucleic acids that are found within the sample. Nucleic acids can be isolated from all biological sample types using methods according to the invention, including animal, plant, fungal, bacterial and viral sources. This method can be used with various volumes of obtained biological samples, and the size of the apparatus can be scaled up or down appropriately to the amount of original sample present and the desired recovery amount of purified nucleic acid product. The apparatus size, for example, can typically be about 4 cm to 50 cm in length, or larger if desired, but is preferably sized comfortably for hand-held or benchtop use. Preferably, biological sample volumes are from about 0.1 mL to about 5 mL of fluid before exposure to any processing materials, buffers, reagents, etc.

Typically, freeing the nucleic acid involves lysing the cells, organelles, membranes and/or structures in which the nucleic acid molecules are typically contained. Lysing can involve mechanical means that physically ruptures the surrounding structure(s) such as, without limitation, homogenization, freeze/thaw, manual grinding or sonication, or a combination thereof, or chemical means, which alters the chemistry of the environment the surrounding structure is in, such as, without limitation, lytic enzymes, for example, lysins, labiase, lysozyme, zymolase, achromopeptidase, mutanolysin, lyostaphin, β-glucanase, α-Hemolysin, streptolysin O, tetanolysin, pectinase, pectolyase cellulose, proteinase, chitinase, lyticase, etc. or detergents or protein denaturants, for example, without limitation, anionic detergents, such as lithium dodecyl sulfate (LDS), sodium dodecyl sulfate (SDS), cationic detergents, nonionic detergents, and zwitterionic detergents, etc. or a combination thereof; or a combination both mechanical and chemical means. Collected biological samples may be frozen and a mortar and pestle or bag and hammer may be used to initiate the lysing process. Typically, lysing also involves several other components, such as adding an RNase or DNase inhibitor so as to help preserve the nucleic acid and prevent degradation of the nucleic acid. Preferably, the sample is associated with a composition including one or more of the following ingredients: chaotropes, detergents, reducing agents, chelators, buffers, or a combination thereof. Lysing may occur before charging of the extraction apparatus or carrying out the methods of the invention described herein, or it may occur within one or more portions of the extraction apparatus and as a part of the inventive methods.

Once the nucleic acid has been freed of its surrounding structure(s) through any conventional technique available to one of ordinary skill in the art, such as by mechanical or chemical means, the nucleic acid needs to be separated from the rest of the lysed biological sample using the extraction apparatus and/or methods according to the invention. A preferred embodiment of the invention includes a solid phase extraction in which the mobile phase, or lysed biological sample, is passed through and over the stationary phase in order to separate the mixture. In one preferred embodiment, at least a portion of the prepared sample suspected of containing nucleic acids is passed through a filtration zone by applying, e.g., pressure to the sample. Preferably, the pressure is not created by a vacuum and instead the pressure is exerted by the displacement of air in association with, or behind, the liquid to force it to enter the filtration zone. In other varying embodiments, at least about a third, preferably at least about fifty percent, more preferably, at least about seventy-five percent, of the sample is dispensed through the filtration zone at least twice, preferably three times, more preferably four times, prior to washing and eluting the nucleic acid. The filtration zone may be equilibrated with a solvent or solution before application of the sample. Once the sample enters the filtration zone, a portion of the nucleic acid in the sample will preferably be retained or adsorbed by the stationary phase and the rest of the sample including, but not limited to, the solvent, salts, denatured proteins, lipids, other nucleic acids and other impurities will pass through. Typically, most, and preferably, at least substantially all, of the freed nucleic acid is adsorbed or bound in the filtration zone.

The filtration zone is typically then washed to remove one or more impurities therefrom. Any conventional wash solution or buffer can be used. Typical wash buffers can include, but are not limited to, ethanol, methanol, Tris, isopropanol, acetone, tris-HCl, ethylenediaminetetraacetic acid (EDTA), etc. The wash buffer can include components of the lysis buffer as well. The filtration zone preferably should be washed at least once, preferably two to three times and in some embodiments, more preferably, four to five times, to remove any remaining impurities. Typically, wash recovery volumes vary with the number of washings, but recovery volumes are most often the volume of the liquid that was put through the filtration zone within about ±5 percent. In one preferred embodiment, the wash buffer is applied in the same manner that the sample was applied, the application of such can be accomplished using the same volume-dispensing mechanism as when the sample was originally applied to the filtration zone.

At least a second portion of the nucleic acid is then eluted from the stationary phase by application of an elution buffer to the filtration zone. Any suitable elution buffer available to those of ordinary skill in the art can be used. Examples of suitable elution buffers include, but are not limited to, tris-HCl, tricine, bicine, TE buffer, water, etc., or a combination thereof. In one preferred embodiment, the elution buffer is applied to the filtration zone in the same manner that the sample and the wash buffer was applied. The same volume-dispensing mechanism may be used, preferably so long as there is no cross-contamination between the elution buffer and any previously applied solution. In a preferred embodiment, some of the nucleic acid is desorbed or debound, more preferably at least substantially all of the nucleic acid is desorbed, upon application of the elution buffer to the filtration zone. Preferably, bound nucleic acid is eluted with about 25 μL to about 1 mL of elution buffer. Preferably, the container of the eluted nucleic acid is sterilized before elution and prior to any further experimentation. The eluted portion of the nucleic acid is then collected for further analysis, diagnosis, or other appropriate use.

Preferably, in one embodiment, the stationary phase, which is associated with the membrane filter includes, or is made of, a silica-based material. In such an embodiment, and due to their hydrophilic properties, nucleic acids get adsorbed onto such a membrane filter with proteins and lipids, which are hydrophobic, and tend not to stick to the membrane. Binding takes place under high salt conditions, typically in the presence of chaotropic salts that remove water from hydrated molecules in solution. Usually, the highest DNA adsorption efficiencies are shown to occur in the presence of buffer solution with a pH at or below the pKa of the surface silanol groups. A possible explanation for this is that the negative charge on the surface of the silica is reduced, which leads to a decrease in the electrostatic repulsion between the negatively charged nucleic acid and the negatively charged silica. The presence of any suitable chaotrope, such as but not limited to guanidinium hydrochloride, guanidine salt, sodium iodide, potassium iodide, sodium thiocyanate, urea, etc., or a combination thereof, causes a salt bridge to be formed between the silica and the nucleic acid backbone. The bound nucleic acid is then washed with a high salt buffer so that the nucleic acid remains adsorbed to the silica-based matrix. Elution takes place preferably under low or no-salt conditions.

In another embodiment, the stationary phase is made of an ion-exchange carrier, preferably an anion-exchange carrier, which is able to reversibly bind to nucleic acids. Adsorption is dependent on the electrostatic interactions between the charged groups of the nucleic acid and the charged groups of the carrier or matrix. Applying a buffer solution that alters the pH and/or ionic strength of the environment the adsorbed nucleic acid is in, alters the binding and allows for elution of the nucleic acid. Exemplary ion-exchange carriers are known in the art and can include diethylamino ethyl (DEAF), DE52, etc. or a combination thereof.

In another embodiment, the stationary phase is a silica-based matrix modified by an anion exchange material.

In one embodiment, the entire extraction of the nucleic acid can take place at or about ambient temperature. This would include the lysis and extraction washes, buffers and elution materials. Preferably, the extraction takes place at about ambient temperatures, preferably about 0° C. to about 60° C., more preferably at temperatures from about 10° C. to about 40° C. In preferred embodiments, the extraction takes place at about 15° C. to 25° C.

Preferably, the eluted purified nucleic acid is at least substantially biologically pure. Once eluted, the purified nucleic acid is typically suitable for any conventional molecular biological applications including, without limitation, ligation, amplification, restriction digestion, recombination, labeling and fluorescent and radioactive sequencing, or any combination thereof. Therefore, the method could further include detecting and/or characterizing the nucleic acid. These processes can more specifically include, but are not limited to, the following biotechniques: genetic fingerprinting; Southern hybridization; Northern hybridization; amplified fragment length polymorphism (AFLP) polymerase chain reaction (PCR); restriction fragment length polymorphism analysis (RFLP); allele-specific oligonucleotide analysis (ASOA); microsatellite analysis; variable number of tandem repeats (VNTR) PCR; dot-blot hybridization; quantitative real-time PCR; polymerase cycling assembly (PCA); nested PCR; quantitative PCR (Q-PCR); asymmetric PCR; DNA footprinting; single nucleotide polymorphism (SNP) genotyping; reverse transcription PCR (RT-PCR); multiplex PCR (m-PCR); multiplex ligation-dependent probe amplification (MLPA); ligation-mediated PCR (LmPCR); methylation specific PCR (MPCR); helicase-dependent amplification (HDA); overlap-extension PCR (OE-PCR); whole-genome amplification (WGA); plasmid isolation; allelic amplification; site-directed mutagenesis; high-throughput genetic screening; or the like, or any combination thereof

Real-time PCR and FRET methodologies have been well described in the literature (see, for example, U.S. Pat. Nos. 4,996,143, 5,565,322, 5,849,489, 6,162,603, each of which is specifically incorporated herein in its entirety by express reference thereto). The LightCycler® platform represents a significant breakthrough in genetic mutation screening and analysis. This system incorporates a rapid, air-driven thermal cycling instrument that can perform 30 polymerase chain reaction (PCR) cycles in less than 20 minutes. It utilizes an in-line microvolume fluorimeter to detect and quantitate fluorescently-labeled hybridization probes, and provides the data necessary for determination of melting curve analyses. The LightCycler® platform provides innovative instrumentation to facilitate the development of genetic analysis tools, and to provide a rapid, qualitative method for the assay of specific nucleotide sequences, and genetic mutations. Detailed application of the instrumentation in amplification and detection methods may be found on the manufacturer's website, and in product application manuals. This technology has also been described, including for example PCT Intl. Appl. Publ. Nos. WO 97/46707, WO 97/46714 and WO 97/46712 (each of which is specifically incorporated in its entirety by express reference thereto). Each of these post-elution techniques can be applied to purified nucleic acids or other structures of interest isolated with the extract apparatus or methods, or both, according to the presently described invention.

The invention also provides for field extraction kits for isolating a structure of interest from a sample that includes the extraction vessel, a collection device to collect one or more samples, and a plurality of reagents necessary to extract the structure of interest from the sample. In one embodiment, the kit provides for isolating a nucleic acid from a sample, such as a biological sample. The kit can further include instructions on performing the method of use. In one aspect, the kit contains solutions or reagents useful in performing the method of use. For example, in one embodiment, the kit includes one or more of the following reagents: chaotropes, detergents, reducing agents, chelators, buffers, or a combination thereof In preferred embodiments, the reagents are self-contained in that they are pre-packaged in individual vessels or tubes either individually, or in combinations, that are ready to be used in performance of the extraction. Preferably, these self-contained reagents are at least sufficiently sterile, and a preferably at least of a pharmaceutically acceptable purity prior to use. In some embodiments, these reagents are contained in one or more replaceable cartridges. In some embodiments, the kit includes components useful for further processing or detecting of the nucleic acid. For example, in a particular aspect, the kit includes one or more of the following components: oligonucleotides that hybridize to the nucleic acid of interest, free deoxyribonucleotide triphosphates and/or a polymerase, or any combination thereof.

The following examples are included to demonstrate illustrative embodiments of the invention. It should be appreciated by those of ordinary skill in the art that the techniques disclosed in the examples that follow represent techniques discovered to function well in the practice of the invention, and thus can be considered to constitute preferred modes for its practice. However, those of ordinary skill in the art should, in light of the present disclosure, appreciate that many changes can be made in the specific embodiments which are disclosed and still obtain a like or similar result without departing from the spirit and scope of the invention.

### Example 1

### Analysis of RNA Extraction of H3N2 Influenza from Human Clinical Sample

A single channel micropipette with a 200 to 1000 μl range commercially available from Pipetman P1000 (Gilson, Middleton, Wis., USA) that operates by piston-driven air displacement, was used as the volume-dispensing mechanism. A sterilized, plastic O-ring was placed on either side of a borosilicate glass fiber disc (Advantec MFS, Inc., Pleasanton, Calif.). The self-sealing aerosol resistant barrier was removed from an injection molded plastic disposable pipette tip in a sterile manner. The disc and the surrounding O-rings were placed nearest the liquid contacting end in the tip so that the disc spanned the width of the pipette tip. The aerosol resistant barrier was replaced into its approximate original location and the filtration vessel was placed into a rack or stand so that the micropipettor could be attached to it without touching it.

A human clinical nasal wash sample that previously tested positive for Influenza Virus subtype H3N2 and was stored in a lysis medium in a microcentrifuge tube (Eppendorf, Hamburg, Germany) at 4° C. for about 1 minute was then pipetted into the embodiment of the extraction vessel as depicted in FIG. 3. The lysis solution was a proprietary solution called PrimeStoreTM and has been described previously in co-pending and commonly owned U.S. Publication No. 2009/0233309, which is hereby incorporated herein by express reference thereto. About 0.4 mL of lysed solution was pipetted through the membrane filter seven times.

After pipetting the sample, the portion of the sample that was not bound to the borosilicate membrane filter and was in a collection container was discarded. Although optional, the membrane was then washed by pipetting up and down a wash buffer (Ambion Wash Buffer I) into a separate collection container. An elution buffer, 100 μL of PCR grade water, was pipetted up and down from another collection container so as to release a portion of the influenza RNA that was bound to the membrane.

rRT-PCR amplification was performed on the eluted RNA in a single-step, single-reaction-vessel reaction-vessel format. Using the UltraSense Platinum One-Step Quantitative RT-PCR System (Invitrogen, Carlsbad, Calif., USA), 2 pi RNA was added to 18 pi master mix containing the following components at the indicated final concentrations: 1× reaction buffer, 1× enzyme mixture containing 500 nM of each Influenza A primer and 300 nM Influenza A probe labeled at the 5′-end with 6-carboxyfluorescein (FAM) reporter dye and at the 3′-end with a nonfluorescent quencher and minor groove binder. Thermocycling was carried out as follows: 30 minutes at 45° C. and 2 minutes at 95° C. for reverse transcription (RT) and denaturation, respectively, followed by 40 amplification cycles consisting of 95° C. for 5 seconds (denaturation) and 60° C. for 30 seconds (extension).

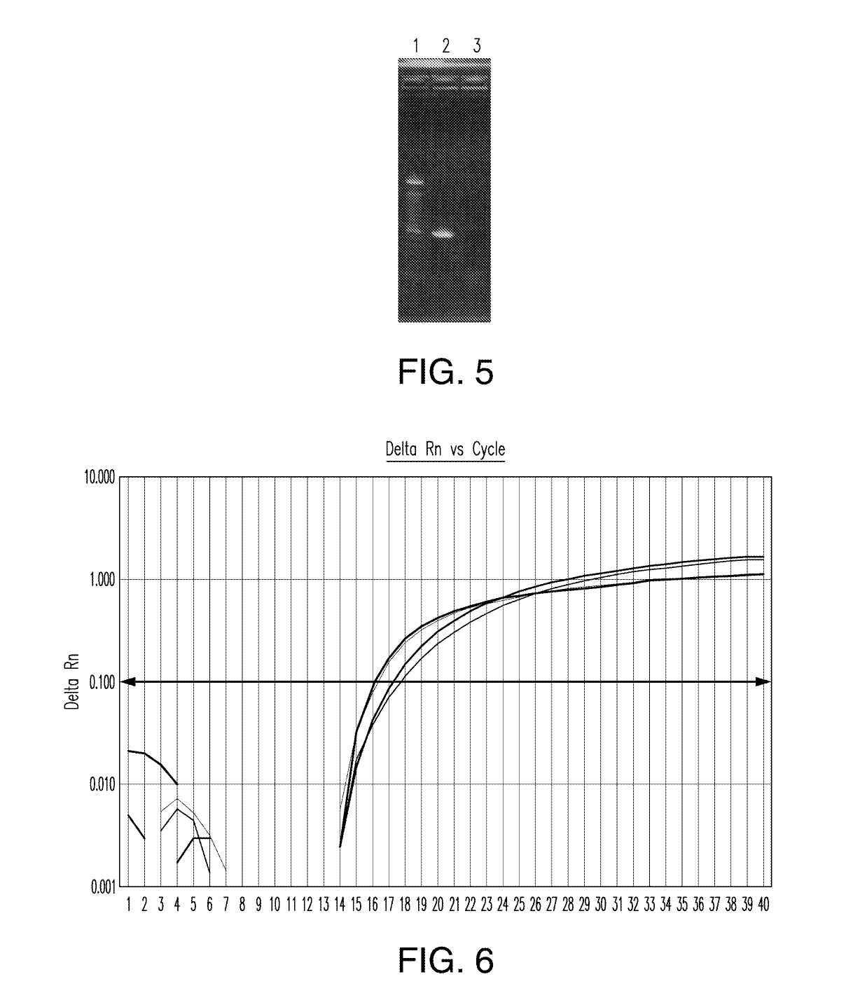

PCR reaction product (5 μl) (lane 2), a ladder (lane 1) and no template control (NTC) (lane 3) were subjected to analytical electrophoresis on 2% pre-cast gels containing ethidium bromide (Invitrogen), as shown in FIG. 5. Lane 2 demonstrated the ability of RNA to be extracted without contamination using a method and apparatus of the present invention. Lane 2 is the resultant PCR amplicon showing that the influenza RNA from the extraction device was of sufficient quality and quantity for nucleic acid testing (NAT).

### Example 2

### Comparison of An Inventive Nucleic Acid Extraction to Prior Art

The rRT-PCR data in FIG. 6 illustrate the ability of a far simpler, more elegant, more inexpensive apparatus and method of the present invention to obtain similar extraction efficiencies, expressed as C_{T} scores, to that of a commercially available extraction columns and methods (RNAqueous™, Ambion). In FIG.6, “delta Rn” represents the fluorescent reporter signal minus a baseline amount. The apparatus as diagrammed in FIG. 3 was used. Both apparatuses used glass-fiber filters to bind RNA in the presence of chaotropic salts. Duplicate extractions of 100 μl samples of influenza A (H1N1) 2009 virus stock (titer=10⁵TCID₅₀/mL) stored in PCR grade water for each apparatus were used. Microcentrifugation was used with the commercial apparatus to drive solutions including the sample, wash, and elution buffer, through the column. The same extraction and lysis media were used for both apparatuses. Extraction efficiency was evaluated using the ABI 7500 sequence detection system with the comparative threshold (C_{T}) method. Influenza A primers and probes were used during the PCR. The average C_{T} score and viral copies detected using the apparatus and methods of the present invention (C_{T}=17.3) negligibly varied from the C_{T} score obtained using the commercial extraction column (C_{T}=16.3).

### Example 3

### Comparison of Nucleic Acid Yield

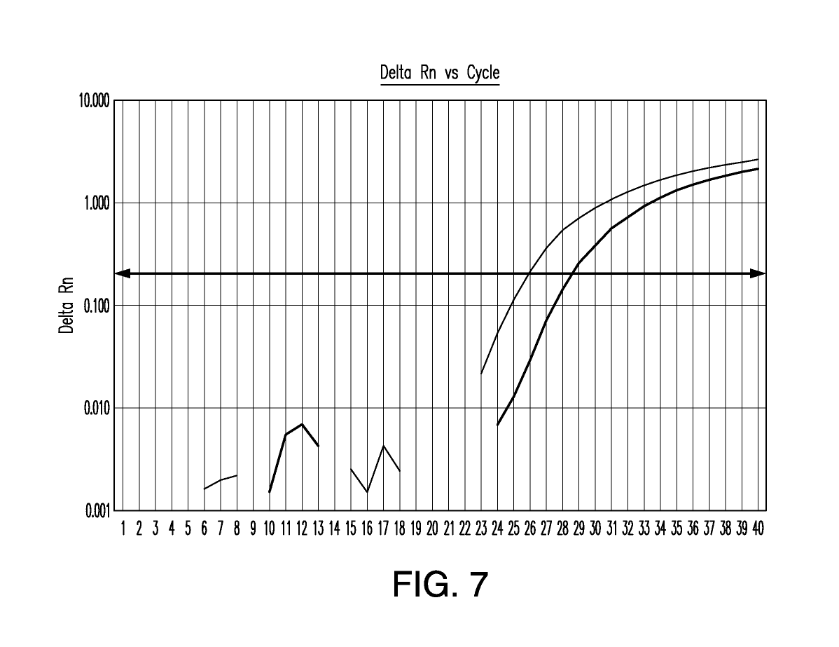

The rRT-PCR data in FIG. 7 illustrate the ability of the apparatus and methods of the present invention to obtain increased nucleic acid yield when increasing the number of times the sample is passed over the membrane filter. In FIG. 7, “delta Rn” represents the fluorescent reporter signal minus a baseline amount. Two separate apparatuses depicted in FIG. 2 were used to extract RNA from two 100 μl samples of viral RNA from a cultured stock of influenza A (H3N2) (titer=10³TCID₅₀/mL). A 10 mL disposable syringe (Becton, Dickinson and Company, N.J.)was used as the volume-displacing mechanism according to the invention. Both apparatuses used glass-fiber filters to bind RNA in the presence of chaotropic salts. One of the samples was drawn up into the syringe, the syringe was attached to the rest of the extraction vessel, and the plunger was pushed once so that the sample was run over the membrane filter once and the contents of the sample that did not bind to the membrane filter were deposited into a collection vessel which was then disposed of. The other sample was drawn up into another syringe, the syringe was attached to the rest of the extraction vessel and the plunger of the syringe was pushed so that the contents of the sample that did not bind to the membrane filter were deposited into a collection vessel and then the plunger was drawn up and pushed three more times while the tip was submersed in the sample present in the collection vessel. The two membrane filters were washed and eluted as described above. Influenza A primers were used on the extracted and filtered samples. The C_{T} score of the sample that had been passed multiple times over the membrane filter showed dramatically increased nucleic acid recovery (C_{T}=26) as when compared with the single passage of the sample over the membrane filter (C_{T}=28.8).

### Example 4

### Use of an Internal Positive Control (“IPC”)

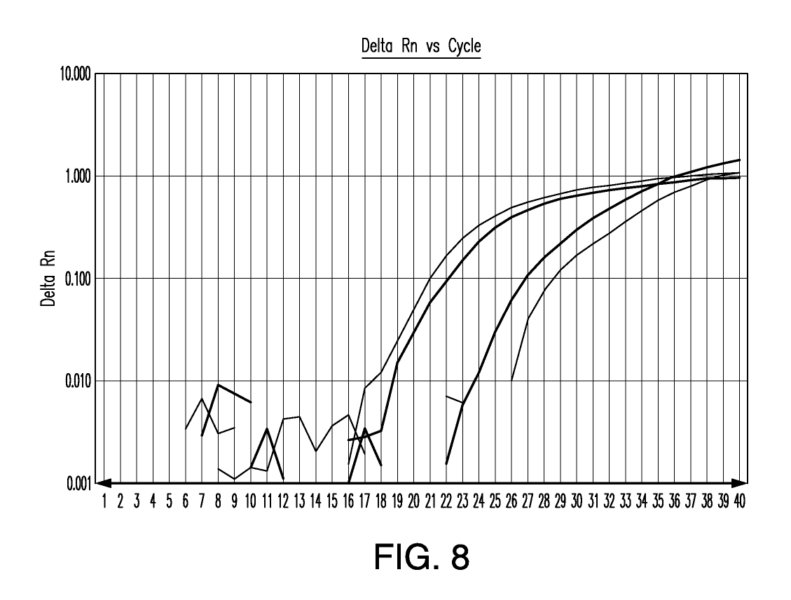

The rRT-PCR data in FIG. 8 illustrate the ability of the apparatus and methods of the present invention to use an internal positive control (“IPC”) to monitor RNA extraction, rRT-PCR and detection of false negative results. In FIG. 8, “delta Rn” represents the fluorescent reporter signal minus a baseline amount. The apparatuses, as depicted in FIG. 2, used glass-fiber filters to bind RNA in the presence of chaotropic salts and a 10 mL syringe as the volume displacing mechanism. Duplicate extractions of 100 μl samples of influenza A (H3N2) virus stock (titer=10³TCID₅₀/mL) stored in PCR grade water for each apparatus were used. The C_{T} score of the IPC (C_{T}=21.4 and 28.1) demonstrates that nucleic acids can be extracted from biological samples with IPC methods so as to help ascertain false positive results.

### Example 5

### Analysis of RNA Extraction of H1N1 Influenza Using an Inventive Embodiment

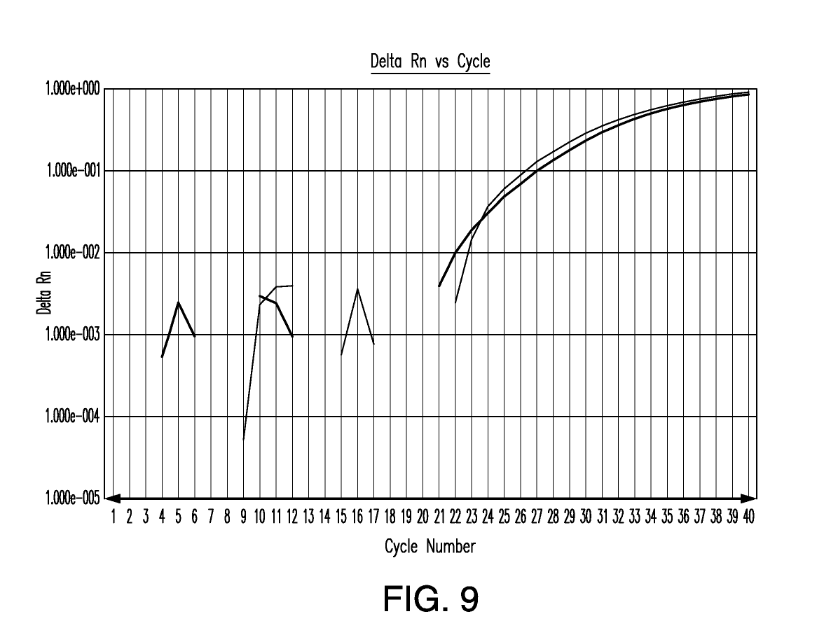

The rRT-PCR data in FIG. 9 illustrate the ability of the apparatus and methods of the present invention to extract viral RNA by using an embodiment of the invention as depicted in FIG. 1. In FIG. 9, “delta Rn” represents the fluorescent reporter signal minus a baseline amount. The apparatus, as depicted in FIG. 1, used glass-fiber filters to bind RNA in the presence of chaotropic salts and a 10 mL syringe as the volume displacing mechanism. Duplicate extractions of 100 μl samples of an Influenza A, subtype H1N1, virus stock (titer=10⁵TCID₅₀/mL) stored in PCR grade water for each apparatus were used. The average C_{T} score (C_{T}=26.8) demonstrates that successful extraction can occur using the apparatus as depicted in FIG. 1, which prevented sample backflow and allowed for flow of the sample across the membrane filter only once.

In accordance with long-standing patent law convention, the words “a” and “an” when used in this application, including the claims, denotes “one or more” unless otherwise specified.

The terms “about” and “approximately,” as used herein, are interchangeable, and should generally be understood to refer to both numbers in a range of numerals. For example, “about 1 to 10” should be understood as “about 1 to about 10.” Moreover, all numerical ranges herein should be understood to include each whole integer within the range, i.e., 1, 2, 3, 4, 5, 6, 7, 8, 9, and 10.

As used herein, the term “biological molecule” refers to any molecule found within a cell or produced by a living organism, including viruses. This may include, but is not limited to, nucleic acids, proteins, carbohydrates, and lipids. As used herein, a “cell” refers to the smallest structural unit of an organism that is capable of independent functioning and is comprised of cytoplasm and various organelles surrounded by a cell membrane. This may include, but is not limited to, cells that function independently, such as bacteria and protists, or cells that live within a larger organism such, as leukocytes and erythrocytes. As defined herein, a cell may not have a nucleus, such as a mature human red blood cell.

As used herein, the term “buffer” includes one or more compositions, or aqueous solutions thereof, that resist fluctuation in the pH when an acid or an alkali is added to the solution or composition that includes the buffer. This resistance to pH change is due to the buffering properties of such solutions, and may be a function of one or more specific compounds included in the composition. Thus, solutions or other compositions exhibiting buffering activity are referred to as buffers or buffer solutions. Buffers generally do not have an unlimited ability to maintain the pH of a solution or composition; rather, they are typically able to maintain the pH within certain ranges, for example, from a pH of about 5 to 7.

The term “chaotrope” or “chaotropic agent” as used herein, includes substances that cause disorder in a protein or nucleic acid by, for example, but not limited to, altering the secondary, tertiary, or quaternary structure of a protein or a nucleic acid while leaving the primary structure intact.

The terms “e.g.,” and “i.e.” as used herein, is used merely by way of example, without limitation intended, and should not be construed as referring only those items explicitly enumerated in the specification.

The phrases “isolated” or “biologically pure” refer to material that is preferably at least substantially, or essentially, free, or more preferably entirely free, from other components that would interact or interfere with the structure of interest, for example, components that normally accompany the material as it is found in its native state. Thus, isolated nucleic acids in accordance with the invention preferably do not contain significant quantities, or any, materials normally associated with the peptides in their in situ environment.

As used herein, the term “nucleic acid” includes one or more types of: polydeoxyribonucleotides (containing 2-deoxy-D-ribose), polyribonucleotides (containing D-ribose), and any other type of polynucleotide that is an N-glycoside of a purine or pyrimidine base, or modified purine or pyrimidine bases (including abasic sites). The term “nucleic acid,” as used herein, also includes polymers of ribonucleosides or deoxyribonucleosides that are covalently bonded, typically by phosphodiester linkages between subunits, but in some cases by phosphorothioates, methylphosphonates, and the like. “Nucleic acids” include single- and double-stranded DNA, as well as single- and double-stranded RNA. Exemplary nucleic acids include, without limitation, gDNA; hnRNA; mRNA; rRNA, tRNA, micro RNA (miRNA), small interfering RNA (siRNA), small nucleolar RNA (snORNA), small nuclear RNA (snRNA), and small temporal RNA (stRNA), and the like, and any combination thereof.

As used herein, the terms “protein,” “polypeptide,” and “peptide” are used interchangeably, and include molecules that include at least one amide bond linking two or more amino acid residues together. Although used interchangeably, in general, a peptide is a relatively short (e.g., from 2 to about 100 amino acid residues in length) molecule, while a protein or a polypeptide is typically a relatively longer polymer (e.g., 100 or more residues in length). However, unless specifically defined by a chain length, the terms peptide, polypeptide, and protein are used interchangeably.

As used herein, “sample” includes anything containing or presumed to contain a structure of interest. Such a structure may be a composition of matter containing one or more nucleic acids, proteins, or other molecule(s) of interest. The term “sample” can thus encompass a solution, cell, tissue, or population of one of more of the same that includes a population of nucleic acids (genomic DNA, cDNA, RNA, protein, other cellular molecules, etc.). The term “sample” and “specimen” are used interchangeably herein in a broad sense, and are intended to encompass a variety of sources that contain nucleic acids, protein, one or more other components of interest, or any combination thereof. Exemplary biological samples include, but are not limited to, whole blood, plasma, serum, sputum, urine, stool, white blood cells, red blood cells, buffy coat, swabs (including, without limitation, buccal swabs, throat swabs, vaginal swabs, urethral swabs, cervical swabs, rectal swabs, lesion swabs, abscess swabs, nasopharyngeal swabs, and the like), urine, stool, sputum, tears, mucus, saliva, semen, vaginal fluids, lymphatic fluid, amniotic fluid, spinal or cerebrospinal fluid, peritoneal effusions, pleural effusions, exudates, punctates, epithelial smears, biopsies, bone marrow samples, fluid from cysts or abcess contents, synovial fluid, vitreous or aqueous humor, eye washes or aspirates, pulmonary lavage or lung aspirates, and organs and tissues, including but not limited to, liver, spleen, kidney, lung, intestine, brain, heart, muscle, pancreas, and the like, or any combination thereof. In some embodiments, the sample may be, or be from, an organism that acts as a vector, such as a mosquito, or tick, or other insect(s).

Tissue culture cells, including explanted material, primary cells, secondary cell lines, and the like, as well as lysates, homogenates, extracts, or materials obtained from any cells, are also within the meaning of the term “sample,” as used herein. Microorganisms (including, without limitation, prokaryotes such as the archaebacteria and eubacteria; cyanobacteria; fungi, yeasts, molds, actinomycetes; spirochetes, and mycoplasmas); viruses (including, without limitation the Orthohepadnaviruses [including, e.g., hepatitis A, B, and C viruses], human papillomavirus, Flaviviruses [including, e.g., Dengue virus], Lyssaviruses [including, e.g., rabies virus], Morbilliviruses [including, e.g., measles virus], Simplexviruses [including, e.g., herpes simplex virus], Polyomaviruses, Rubulaviruses [including, e.g., mumps virus], Rubiviruses [including, e.g., rubella virus], Varicellovirus [including, e.g., chickenpox virus], rotavirus, coronavirus, cytomegalovirus, adenovirus, adeno-associated virus, baculovirus, parvovirus, retrovirus, vaccinia, poxvirus, and the like), algae, protozoans, protists, plants, bryophytes, and the like, or any combination of any of the foregoing, that may be present on or in a sample are also within the scope of the invention, as are any materials obtained from clinical or forensic settings that contain one or more nucleic acids are also within the scope of the invention. The ordinary-skilled artisan will also appreciate that lysates, extracts, or materials obtained from any of the above exemplary biological samples are also within the scope of the invention.

Samples in the practice of the invention can be used fresh, or can be used after being stored for a period of time, or for an extended period of time, including for example, cryopreserved samples and the like, and may include material of clinical, veterinary, environmental or forensic origin, may be isolated from food, beverages, feedstocks, potable water sources, wastewater streams, industrial waste or effluents, natural water sources, soil, airborne sources, pandemic or epidemic populations, epidemiological samples, research materials, pathology specimens, suspected bioterrorism agents, crime scene evidence, and the like. Preferably, the samples are used before substantial degradation occurs in the structure of interest therein.

The term “recombinant” indicates that the material (e.g., a polynucleotide or a polypeptide) has been artificially or synthetically (non-naturally) altered by human intervention. The alteration can be performed on the material within, or removed from, its natural environment or state. Specifically, e.g., an influenza virus is recombinant when it is produced by the expression of a recombinant nucleic acid. For example, a “recombinant nucleic acid” is one that is made by recombining nucleic acids, e.g., during cloning, DNA shuffling or other procedures, or by chemical or other mutagenesis; a “recombinant polypeptide” or “recombinant protein” is a polypeptide or protein which is produced by expression of a recombinant nucleic acid; and a “recombinant virus,” e.g., a recombinant influenza virus, is produced by the expression of a recombinant nucleic acid.

The term “substantially free” or “essentially free,” as used herein, typically means that a composition contains less than about 10 weight percent, preferably less than about 5 weight percent, and more preferably less than about 1 weight percent of a compound. In a preferred embodiment, these terms refer to less than about 0.5 weight percent, more preferably less than about 0.1 weight percent or even less than about 0.01 weight percent. The terms encompass a composition being entirely free of a compound or other stated property, as well. With respect to degradation or deterioration, the term “substantial” may also refer to the above-noted weight percentages, such that preventing substantial degradation would refer to less than about 15 weight percent, less than about 10 weight percent, preferably less than about 5 weight percent, etc., being lost to degradation. The term “entirely free” means no more than a trace amount of such undesired material(s) are present, e.g., as an impurity in the extraction apparatus or a portion thereof.

The term “structure” or “structures,” as used herein, generally refers to any type of matter or substance including, but not limited to, one or more molecules, elements, ions, compounds, or any combination thereof. The term “analyte” or “component” may be used interchangeably with “structure.” The term “structure” can be used to denote one or multiple structures that can, for example, bind or adsorb, to the filtration membrane at the same time. This does not, however, mean that the structures have to elute at the same time when exposed to substantially the same elution conditions.

The foregoing detailed description outlines features of several embodiments so that those of ordinary skill in the art may better understand the various aspects of the present disclosure describing the invention. Those of ordinary skill in the art should appreciate that they may readily use the present disclosure as a basis for designing or modifying other apparatus and method details for carrying out the same purposes and/or achieving the same advantages of the embodiments introduced herein. Those of ordinary skill in the art should also realize that such equivalent details do not depart from the spirit and scope of the present disclosure, and that they may make various changes, substitutions and alterations herein without departing from the spirit and scope of the present disclosure. In the drawings, the same or similar elements are denoted by the same or similar reference numerals even though they are depicted in different figures.

What is claimed is:

1. An extraction apparatus comprising:
a filtration vessel that has at least one receiving end and that comprises a membrane filter adapted to bind a structure of interest thereto, wherein the membrane filter is disposed at least substantially across a width of the filtration vessel and at least partially therein;
a volume-dispensing mechanism adapted to controllably dispense and forcibly inject an amount of liquid operably associated with the filtration vessel to filter the liquid therethrough; and
a collection container adapted to receive the filtered liquid.

2. The apparatus of claim 1, wherein the structure of interest is a nucleic acid.

3. The apparatus of claim 2, further comprising an aerosol-resistant barrier disposed on at least one side of the membrane filter.

4. The apparatus of claim 1, further comprising a flow valve positioned between the volume-dispensing mechanism and the filtration vessel or within the filtration vessel to limit fluid flow to one direction.

5. The apparatus of claim 1, further comprising a substantially leak-free connector positioned between the volume-dispensing mechanism and the membrane filter.

6. The apparatus of claim 2, wherein the membrane filter comprises a polymer-based or silica-based matrix.

7. The apparatus of claim 6, wherein the matrix comprises silica fiber; silica gel; silica resin; silica-cellulose; silica dioxide; an ion-exchange resin; borosilicate; glass; or diatomaceous earth, or a combination or a blend thereof.

8. The apparatus of claim 7, wherein the ion-exchange resin comprises a polymer comprising diethylaminoethyl-, quaternary aminoethyl- or quaternary ammonium-groups.

9. The apparatus of claim 2, wherein the filtration vessel comprises polyethylene, linear polyethylene, low density polyethylene, polypropylene, polystyrene, polycarbonate, endotoxin-free or pyrogen-free plastic, or a combination or a blend thereof.

10. The apparatus of claim 1, wherein the liquid dispensing mechanism comprises a piston pump, syringe, pipette, micropipette, bulb, or dropper.

11. The apparatus of claim 1, wherein the membrane filter is restricted from movement by a tether comprising at least one gasket, O-ring, indentation of an opposing inner surface of the filtration vessel, adhesive, or a frit, or a combination thereof.

12. The apparatus of claim 1, wherein the collection container releasably attaches at an end of the filtration vessel.

13. The apparatus of claim 1, further comprising a replaceable cartridge that holds the amount of liquid operably associated with the volume dispensing mechanism and the filtration vessel.

14. The apparatus of claim 13, wherein the replaceable cartridge is adjacent a flow path from the volume dispensing mechanism to the filtration vessel.

13. A method for extracting a structure of interest from a liquid, which comprises:
preparing a sample suspected of containing a structure of interest for passage through a filtration zone; and
dispensing a portion of the prepared sample through a filtration zone by applying and controlling injection pressure applied to the sample.

16. The method of claim 15, further comprising washing the filtration zone to remove one or more impurities therefrom.

17. The method of claim 16, further comprising eluting at least a portion of the structure of interest from the filtration zone; and collecting the portion of the structure of interest in a collection zone.

18. The method of claim 16, wherein the sample is a biological sample comprising blood, plasma, cell, tissue, or serum, or any combination thereof.

19. The method of claim 18, wherein the structure of interest is a nucleic acid.

20. The method of claim 19, wherein at least a portion of the sample is dispensed through the filtration zone at least twice prior to the washing and eluting.

21. The method of claim 19, which further comprises sterilizing the collection zone after collecting the eluted second portion of the nucleic acid.

22. The method of claim 19, which further comprises preparing the sample for dispensing by first associating the sample with a composition comprising one or more:
chaotropes, detergents, reducing agents, chelators, buffers, or a combination thereof.

23. The method of claim 19, wherein preparing the sample includes lysing the sample suspected of containing the nucleic acid.

24. The method of claim 19, which further comprises washing the filtration zone at least twice before eluting the second portion of the nucleic acid therefrom.

25. The method of claim 19, wherein the eluting comprises applying TE buffer, water, tricine, bicine, or Tris-HC1, or a combination thereof.

26. The method of claim 19, wherein the extracting is performed at temperatures from about 10° C. to about 40° C.

27. The method of claim 15, wherein the extracting is performed within up to about 1 to 10 minutes of placing the sample in the filtration zone.

28. The method of claim 19, further comprising detecting or characterizing the nucleic acid.

29. The method of claim 15 wherein the portion of the prepared sample is a pre-calibrated unit amount thereof.

30. The method of claim 15, wherein the prepared sample is disposed in a replaceable dispensing zone that is fluidly connected between a dispensing mechanism and the collection zone.

31. The method of claim 15, wherein the dispensing comprises operating a plunger to pressurize the prepared sample.

32. A field extraction kit, in suitable container device, comprising:
(a) the extraction apparatus of claim 1;
(b) a collection device to collect one or more samples; and
(b) self-contained reagents comprising one or more: chaotropes, detergents, reducing agents, chelators, buffers, or a combination thereof.

33. A set of instructions to carry out the method of claim 13 associated with the extraction apparatus operably associated therewith.
